(19) United States
(12) Patent Application Publication
(10) Pub. No.: US 2025/0009251 A1
(43) Pub. Date: Jan. 9, 2025

(54) METHOD FOR MONITORING A RESPIRATORY RATE OF A PERSON

(71) Applicant: E-NOVIA S.P.A., Milano MI (IT)

(72) Inventors: Alessandra ANGELUCCI, Milano MI (IT); Andrea ALIVERTI, Como CO (IT); Dario FROIO, Milano MI (IT); Federico Lorenzo MORO, Milano MI (IT)

(73) Assignee: E-NOVIA S.P.A., Milano MI (IT)

(21) Appl. No.: 18/709,916

(22) PCT Filed: Nov. 17, 2022
(86) PCT No.: PCT/IB2022/061089

§ 371 Date: May 14, 2024

(30) Foreign Application Priority Data
Nov. 18, 2021 (IT) 102021000029204

(51) Int. Cl. A61B 5/08; A61B 5/00; A61B 5/11

(52) U.S. Cl. CPC A61B 5/0816; A61B 5/1118; A61B 5/6823; A61B 5/7225; A61B 5/7267; A61B 2562/0219; A61B 2562/04

(57) ABSTRACT

A method of continuously monitoring the respiratory rate of a person is disclosed. A sensor system using two or three inertial sensors is installed on a person's body, to enhance the accuracy of estimations of respiratory rate in static and dynamic activities. The method includes the steps of determining whether a person's activity in progress is either a static activity or a dynamical activity; and using this information for enhancing the accuracy of estimations of respiratory rate in static and dynamic activities.

### TECHNICAL FIELD

The present disclosure relates to methods for monitoring a person using a wearable device having at least two inertial sensors, and to a related method of continuous monitoring the respiratory rate of the person using the information about the activity in progress.

### BACKGROUND

Respiratory rate is a fundamental factor of prognosis that provides important information on the health of a person. Many pathological conditions of the heart and lungs, in particular pneumonia and cardiac arrest, affect respiratory rate and could be predicted with prolonged monitoring of the respiratory rate.

Despite the relevance of respiratory rate as prognostic factor is widely demonstrated in literature, the current gold standard for measuring the respiratory rate is the number of breaths performed in one minute, identified through auscultation or observation, which is not suitable for prolonged monitoring outside the clinical environment. An alternative to this is the employment of dedicated devices, but a limitation that is found in several studies is that in such studies physiological parameters are usually detected with spot measurements and when the subject is at rest, while it is known that physical activity has an influence on cardiorespiratory function.

The patent application WO2019/012384 discloses a wearable device for continuous monitoring of the respiratory rate of a person, comprising three inertial sensors applied on parts of the body of the person as shown in FIG. 1. Using the signals generated by the three inertial sensors, the prior application provides a method for estimating the respiratory rate.

Even if this prior method provides good estimations of the respiratory rate of a person, there is still the need of improving the accuracy of estimations, in particular while the person is performing dynamic activities.

### SUMMARY

Studies carried out by the inventors have shown that it is possible to improve further the prior method disclosed in the PCT patent application WO2019/012384. In particular, it has been found that it is possible to exploit the sensor system of the above PCT patent application using two inertial sensors only, installed on a person's body, for identifying a person's activity in progress with the method as defined in claim 1. This outstanding result is substantially obtained through the steps of:
determining preliminarily an activity in progress of the person as either a static activity or a dynamical activity, and using this information for enhancing accuracy of estimations of respiratory rate.

Preferred embodiments are defined in the annexed claims.

### BRIEF DESCRIPTION OF THE DRAWINGS

FIG. 1 is a schematic illustration of a dummy wearing a wearable device for continuous monitoring of respiratory rate using three inertial sensors.

FIGS. 2a to 2c illustrate a schematic flowchart of operations carried out for recognizing a human activity using a) one inertial sensor; b) two inertial sensors; c) three inertial sensors.

FIG. 3a illustrates a flowchart of operations of a method of this disclosure of an algorithm for estimating respiratory parameters of a person by filtering the first principal component of signals of inertial sensors installed on the thorax and on the abdomen referred to the reference sensor by applying either a first filter or a second filter depending on the recognized human activity.

FIG. 3b depicts an embodiment of the flowchart of FIG. 3a that illustrates how the preliminary determination of the activity in progress is used to adjust the filtering of the quaternions for determining the respiratory parameters.

FIG. 4 shows schematically the ANT Shared Channel topology implemented for reading the three inertial sensors installed on the person's body as shown in FIG. 1.

FIG. 5 shows schematically a) Block diagram of the software on the master unit; b) Block diagram of the firmware on each slave unit.

FIG. 6 shows five exemplary networks created using deep learning methods for identifying a human activity in progress: a) 1D Convolutional Neural Network (1DCNN); b) 2D Convolutional Neural Network (2DCNN); c) Single-layer Long Short Term Memory (BASE LSTM); d) Multi-layer Long Short Term Memory (MULTI LSTM); e) Gated Recurrent Unit (GRU).

FIG. 7 is a graph of boxplots of the obtained values of respiratory rate (RR) in the different postures and activities. The labels are the following: 1—Sitting; 2—Supine; 3 Prone; 4—Lying Left; 5—Lying Right; 6—Standing; 7—Walking slow; 8—Walking fast; 9—Running; 10—Cycling.

FIG. 8 is a block diagram of another algorithm for estimating respiratory parameters of a person in function of either the first principal component of signals of inertial sensors installed on the thorax and on the abdomen referred to the reference sensor, or the second/higher-order principal component depending on the recognized human activity.

FIG. 9 is a block diagram for a method for estimating respiratory parameters of a person.

FIG. 10 is a graph of the spectrum of the first principal component in a static posture of signals of inertial sensors installed on the thorax and on the abdomen referred to the reference sensor, and the spectrum of the sum of the two signals.

FIG. 11 is a graph of the spectrum of the first principal component during walking activity of signals of inertial sensors installed on the thorax and on the abdomen referred to the reference sensor, and the spectrum of the sum of the two signals.

FIG. 12 is a graph of the spectrum of the second principal component during walking activity of signals of inertial sensors installed on the thorax and on the abdomen referred to the reference sensor, and the spectrum of the sum of the two signals.

### DESCRIPTION OF EXEMPLARY EMBODIMENTS

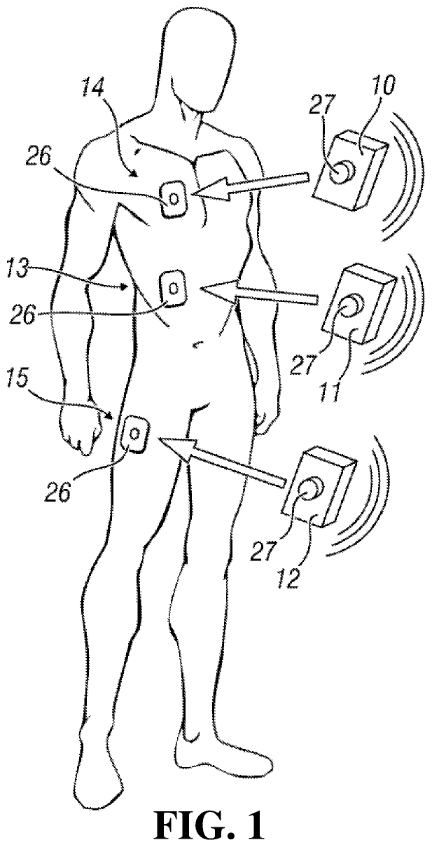

In the ensuing description, the methods of the invention will be illustrated referring mainly to the particular case in which three inertial sensors (hereinafter referred to also as “sensor units”) are installed on the person's body, as shown in FIG. 1, namely two inertial sensors installed respectively on the thorax and on the abdomen, and a reference inertial sensor positioned on a part of the body not subject to respiratory movements, but fixed with respect to the torso, and functioning as reference for the other two inertial sensor. Preferably, the reference inertial sensor may be positioned, for example, at the height of the pelvis (superior iliac spine 15 or else in the proximity of the coccyx), or even on the lower back of the person.

Respiratory parameters are known to change during different activities and in different postures, having a system that combines respiratory parameters and human activity recognition (HAR) would provide even more clinically relevant information.

In the prior sensor system shown in FIG. 1 and disclosed also in [A. Cesarco, S. Gandolfi, I. Pini, E. Biffi, G. Reni, and A. Aliverti, “A novel, low cost, wearable contact-based device for breathing frequency monitoring,” Proc. Annu. Int. Conf. IEEE Eng. Med. Biol. Soc. EMBS. pp. 2402-2405, 2017. doi: 10.1109/EMBC.2017.8037340] [A. Cesareo, E. Biffi, D. Cuesta-Frau, M. G. D'Angelo, and A. Aliverti, “A novel acquisition platform for long-term breathing frequency monitoring based on inertial measurement units,” Med. Biol. Eng. Comput., vol. 58, no. 4, pp. 785-804, 2020, doi: 10.1007/s11517-020-02125-9], two sensor units are aimed at detecting chest wall breathing-related movements, one is located on the thorax and the other on the abdomen; the last IMU is placed on a position not involved in respiratory motion but integral with body movement, most often on the lower back. In fact, the influence of posture on chest wall motion is intensively studied in the literature. It was previously studied that most of the chest wall volume change is distributed in the thoracic compartment in vertical postures and in abdominal compartment in horizontal postures [M. Romei et al., “Effects of gender and posture on thoraco-abdominal kinematics during quiet breathing in healthy adults,” Respir. Physiol. Neurobiol., vol. 172, no. 3, pp. 184-191, 2010, doi: https://doi.org/10.1016/j.resp.2010.05.018]. For this reason, the system's configuration is particularly advantageous for a thorough analysis of the chest movement during breathing in different postures, since it can account for the different contributions of the chest wall in different positions.

From the post-processing side, the algorithm to extract breathing parameters allowed to obtained results that confirm what is known in the literature [A. Angelucci, D. Kuller, and A. Aliverti, “A Home Telemedicine System for Continuous Respiratory Monitoring.,” IEEE J. Biomed. Heal. informatics, vol. 25, no. 4, pp. 1247-1256 April 2021, doi: 10.1109/JBHI.2020.3012621] [W. Qi and A. Aliverti, “A Multimodal Wearable System for Continuous and Real-time Breathing Pattern Monitoring During Daily Activity.,” IEEE J. Biomed. Heal. Informatics, December 2019, doi: 10.1109/JBHI.2019.2963048], i.e. that the frequency is higher in dynamic conditions.

Furthermore, it has to be noted that most systems are not able to provide measurements of the respiratory rate during demanding dynamic activities like running. This system is advantageous both for sports and medical applications, due to its ability to measure this parameter in a broad range of situations.

However, the signal-to-noise ratio is too high while climbing the stairs and the algorithm could not be applied.

The results obtained showed an overall good capability to recognize different activities, independently from the age or the gender of the subjects. Although only features in time and not in frequency were used in the case with three units, the comparison between the use of a single units compared to the use of three, showed that the second works better, with higher accuracy and f1-score both for machine and deep learning methods. A final consideration regards the first step of the data preparation: removing the initial transitory might have led to an overestimation of the accuracy.

The present disclosure presents an advanced prototype suitable for continuous monitoring and shows how to exploit the possibility to perform human activity recognition (HAR) from the raw sensor data.

### II. MATERIALS AND METHODS

### A. Dataset

The 20 healthy subjects involved in the research (9 men, 11 women) were between 23 and 54 years old at the time of the study, with mean age 26.8, mean height 172.5 cm and mean weight 66.9 kg. The experimentation was approved by the Ethical Committee of Politecnico di Milano (Protocol number: 20/2020) and all participants signed an informed consent.

The protocol included seven static postures (sitting with support, sitting without support, supine, prone, left decubitus, right decubitus, standing) and five dynamic activities (walking slow at 4 km/h, walking fast at 6 km/h, running, climbing up and down the stairs, cycling). The walking and running activities were performed on a treadmill, while the cycling activity was performed on an ergometer.

### B. Hardware, Firmware and Data Transmission

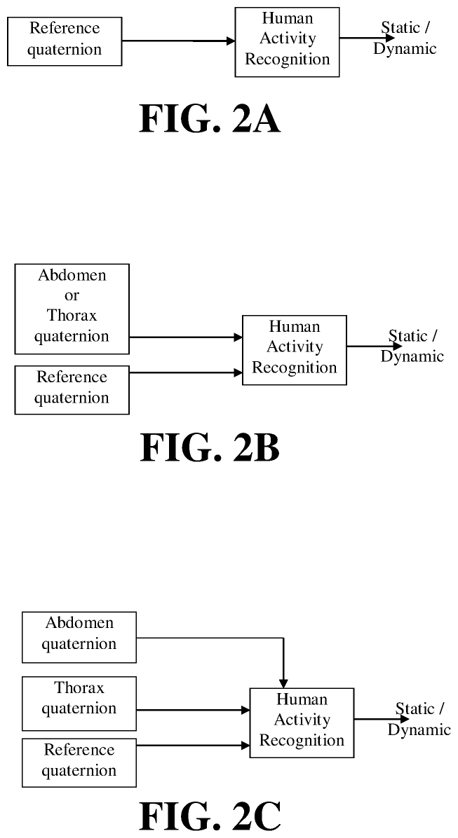

As shown in FIGS. 2a, 2b and 2c, human activity recognition may be performed by processing untreated signals (“raw data”) generated by the inertial sensor used as a reference (FIG. 2a), for example installed on the lower back of a person, or by processing untreated signals generated by the reference inertial sensor and another inertial sensor installed either on the thorax or on the abdomen (FIG. 2b) of the person, or yet by processing untreated signals generated by the reference inertial sensor, the inertial sensor installed on the thorax and the inertial sensor installed on the abdomen (FIG. 2c) of the person. In the ensuing description, reference will be made to a test system configured as in FIG. 2c in which three inertial sensors are installed on the person's body and untreated signals generated by all of them are used to determine the activity in progress, though the same observations hold mutatis mutandis when only the reference sensor (FIG. 2a) or the reference sensor and one other inertial sensor, between the thorax sensor and the abdomen sensor (FIG. 2b), are used.

The sensor system used for the test is composed of three units, one located on the thorax, one on the abdomen and one on the lower back, used as a reference.

Data coming from the three units may be collected either by means of an ANT USB2 Stick that is plugged into a personal computer during the acquisitions or by an Android smartphone that supports ANT.

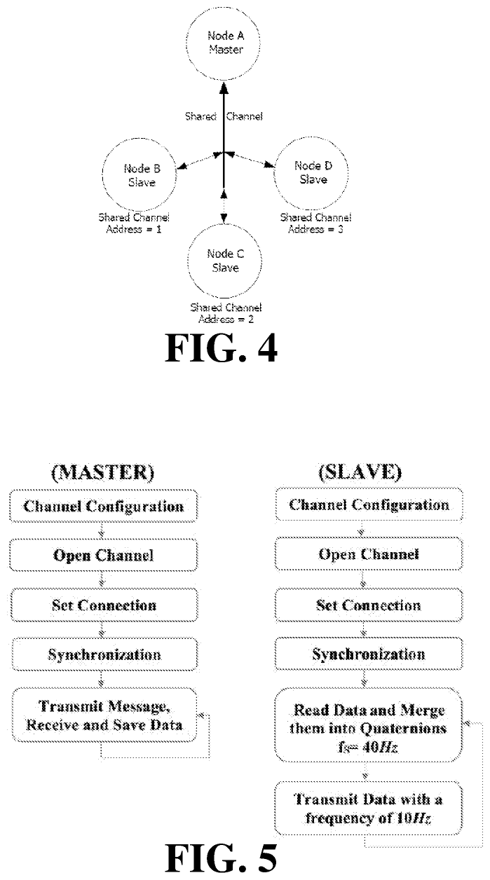

The sensor data are composed of three accelerometer components, three gyroscopes components and three magnetometer components and they are sent to the microcontroller with a 40 Hz rate. The microcontroller, then, computes the 9-axis quaternion, transmitting one quaternion out of four to the USB2 stick through a radio-frequency antenna integrated in each unit, resulting in a 10 Hz frequency, with the ANT communication protocol. The USB2 Stick is the receiver of the data sent by the microcontroller and it is configured as the master, while the three units work as the slaves and they are the transmitters of the sensor's detections. The topology of the network is called Shared Channel and is shown in FIG. 4, while FIGS. 5a and 5b show the block diagrams of the program running on the master device and the firmware of the slave devices, respectively. The master channel has a channel period of 30 Hz, so that it has a 10 Hz time slot to address each of the units (thoracic, abdominal and reference).

The nine components provided by the IMU sensor are calibrated and then used by the microcontroller to calculate a quaternion, which is a four-dimensional complex number [q1 q2 q3 q4]. The fusion of the data collected from the sensor is done by using the sensor fusion iterative algorithm developed by Madgwick et al. [S. O. H. Madgwick, A. J. L. Harrison, and R. Vaidyanathan, “Estimation of IMU and MARG orientation using a gradient descent algorithm,” in 2011 IEEE international conference on rehabilitation robotics, 2011, pp. 1-7.], which is able to compute the quaternions representing the orientation changes of each unit relative to the earth frame. Each component is expressed through a floating-point value, ranging from −1 to 1 and it is transmitted in a byte of the data payload. Moreover, it is also present a counter, increased every four quaternions calculated with a frequency of 40 Hz, in order to identify the n-th transmission.

### C. Respiratory Signal Processing

The process which leads to the respiratory parameters extraction from the data collected by the units is performed offline using a software that implements the previously validated algorithm by Cesareo et al. [A. Cesareo, Y. Previtali, E. Biffi, and A. Aliverti, “Assessment of breathing parameters using an inertial measurement unit (IMU)-based system,” Sensors (Switzerland), vol. 19, no. 1, pp. 1-24, 2019, doi: 10.3390/s19010088.] using the Python programming language, with small variations to adapt the algorithm to the processing of dynamic activities. In particular, the variations are in the cut-off frequencies of some of the implemented filters. The whole elaboration algorithm can be subdivided into four main parts: pre-processing, dimension reduction, spectrum analysis and processing.

In the pre-processing phase, the data divided by unit of origin are organized in four arrays, where missing data are replaced after interpolation is performed. The quaternions are created by combining the arrays. The same selection was done in to train the Human Activity Recognition algorithm that is presented in the next section.

Afterwards, the quaternion product is computed, providing the orientations of the thoracic and abdominal unit referred to the orientation of the reference unit. (1) and (2) show how these computations are performed:

$${}_{Ref}^{Th} \hat{q} = {}_{Earth}^{Th} \hat{q} ⊗ {}_{Earth}^{Ref} \hat{q} *= {}_{Earth}^{Th} \hat{q} ⊗ {}_{Earth}^{Ref} \hat{q} \qquad ( 1 )$$

$${}_{Ref}^{Ab} \hat{q} = {}_{Earth}^{Ab} \hat{q} ⊗ {}_{Earth}^{Ref} \hat{q} *= {}_{Earth}^{Ab} \hat{q} ⊗ {}_{Earth}^{Ref} \hat{q} \qquad ( 2 )$$

As a consequence, the non-respiratory movements are reduced because angular changes are referred to the reference unit, which does not detect breathing-related motions, but it is integral with trunk movement. Then, the baseline is computed by means of the moving average on 97 samples for each quaternion component and subtract to them order to remove the residual non-breathing movement. The generated components are the input for the dimension reduction block.

With the aim of reducing the dimension of the dataset, the Principal Component Analysis (PCA) [S. Wold, K. Esbensen, and P. Geladi, “Principal component analysis,” Chemom. Intell. Lab. Syst., vol. 2, no. 1-3, pp. 37-52, 1987], [M. Ringnér, “What is principal component analysis?,” Nat. Biotechnol., vol. 26, no. 3, pp. 303-304, 2008] is performed. The first component, the one with the greatest amount of variance explained, is computed for the thorax and for the abdomen and considered as respiratory signal and constitute the basis for the spectrum analysis.

The generated signals are filtered with a Savitzky-Golay FIR (Finite Impulse Response) smoothing filter of the 3rd with a window length of 31 samples. This filter works using the linear least squares method in order to fit successive sub-sets of adjacent data with a third order polynomial. In this way, the noise is decreased without changing the shape and the signal peaks height. Then, the mean (fmean) and the standard deviation (fstd) of the inverse of the distances between subsequent peaks are considered to obtain a frequency estimate. fmean and fstd are used to compute fthresh and the procedure is performed both for the thoracic component and the abdominal component as in (3):

$$fthresh = max ( fthresh\_min , fmean - f s td ) \qquad ( 3 )$$

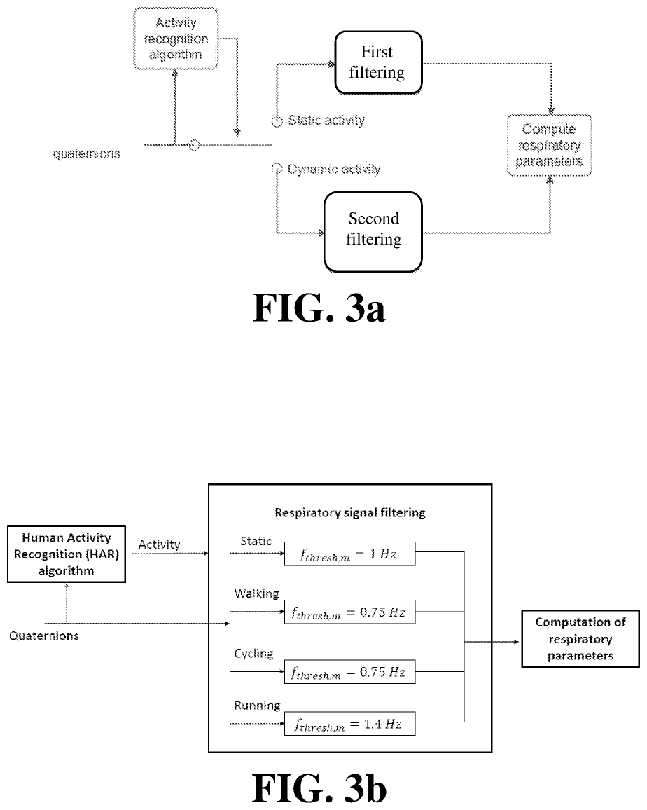

According to an aspect, fthresh_min may be different for static postures and dynamic activities. As schematically shown in FIG. 3a, the first principal component may be filtered with a first filter defined by threshold frequencies fthresh-static if a static activity is determined, and with a second filter defined by threshold frequencies fthresh-dynamic if a dynamic activity is determined.

It is worth remarking that, according to one aspect, the activity in progress is determined preliminarily without calculating a respiratory rate of the person. This determination is obtained by means of a pre-trained activity recognition algorithm receiving as entries at least the reference quaternion of the reference unit. According to an option, the activity in progress is determined with the pre-trained activity recognition algorithm receiving as entries the reference quaternion and also the first quaternion generated by the thoracic or by the abdominal unit.

As illustrated in FIG. 3a, after the activity in progress is identified as either static or dynamic, a first filter or a second filter is chosen accordingly in order to derive respiratory parameters of the patient.

According to an aspect illustrated in FIG. 3b, it is preliminarily determined which activity is in progress without calculating a respiratory rate of the person, then the filtering is carried out with a filter corresponding to the determined activity, for example with a filter having a threshold frequency fthreshm=1 Hz when the patient is performing a static activity, or with a threshold frequency fthreshm=0.75 Hz when the patient is cycling or walking, or with a threshold frequency fthreshm=1.4 Hz when the patient is running.

Once the threshold frequencies of both the thoracic and the abdominal unit are obtained, the low-frequency threshold is computed as the minimum between the abdominal low threshold and the thoracic low threshold. The use of a low threshold helps in the identification of the power spectral density (PSD) peak related to the respiratory rate and does not consider very low frequency peaks, often caused by movement artifacts. Subsequently, the PSD estimate is computed employing the Welch's method, with the Hamming window type, 300 samples as window size, and 50 samples of overlap.

The PSD maximum in the interval between the computed low threshold and a maximum (for example 1 Hz for static postures, 0.75 Hz for walking and cycling, 1.4 Hz for running) is identified (fpeak), which is used to build the adaptive band-pass filter settings (centered in fpeak). In particular the upper and lower cut-off frequencies, for both thorax and abdomen, are obtained as in (4) and (5):

$$f U = f p e a k + 0.04 Hz \qquad ( 4 )$$

$$fL = max ( fthreshm , fpeak - 0.04 Hz ) \qquad ( 5 )$$

The final processing block comprises all the processes intended to extract breathing frequency and other respiratory parameters from the signals obtained after the dimension reduction block.

The first step is the application of the band-pass filter with the previously set cut-off frequencies fU and fL. Since the frequencies are dependent on the fpeak the result is an adaptive filter, based on the specific analysed recording.

Then, a parametric tuning based on the fpeak value is performed. This is necessary for the subsequent steps of filtering and maxima and minima detection. In particular, the involved parameters are the window length in terms of samples for the third order Savitzky-Golay filter and the minimum peak distance. In fact, the algorithm chooses the tallest peak in the signal and ignores all peaks within the decided distance and the minimum prominence threshold, through which is possible to set a measure of relative importance; a more detailed description of the parameters can be found in the work by Cesareo et al. [A. Cesareo, Y. Previtali, E. Biffi, and A. Aliverti, “Assessment of breathing parameters using an inertial measurement unit (IMU)-based system,” Sensors (Switzerland), vol. 19, no. 1, pp. 1-24, 2019, doi: 10.3390/s19010088.]

Afterwards, filtered signals are furtherly smoothed through the application of a third order Savitzky-Golay FIR filter, to optimize subsequent detection of maxima and minima point, which are identified applying the parameters previously set. Moreover, other than the thoracic and the abdominal signal, the process is repeated also for the sum of the two signals, once they were filtered with the Savitzky-Golay filter. The respiratory rate is thus obtained breath-by-breath and the values obtained for each posture or activity are reported in Section 3.

### D. Human Activity Recognition Algorithm

After the window selection, the data processing to train the activity recognition algorithm is different from the one for the respiratory analysis.

The next step involved the creation of a single large dataset containing all the activity properly labelled for all the subjects. In a first phase, it was chosen to run the algorithm on the data of the reference unit, because it can be considered representative of the subject's positions. Secondly, the algorithm was trained on the signals coming from all three units (thoracic, abdomen, and reference). In both cases, a dataset was obtained merging the tasks “sitting without support” and “sitting with support” in a single label called “sitting”, and the tasks “walking at 4 km/h” and “walking at 6 km/h” in a label called “walking”, so the final dataset has 10 labels. This was done to increase the variability of the signal during the training process, so that the algorithm can distinguish a person that sits with a back support from a supine one and between a fast walk and a run.

The resulting dataset was unbalanced, because the labels “sitting” and “walking” had about twice the data of the other labels. The unbalancing was kept in the situation with one unit, while data were balanced in the training with three units. A common balancing procedure was used, which consists in reducing the samples of each label to same number of the activity with the lowest amount of data.

The implemented pre-processing steps were data standardization, label encoding and segmentation. Standardization is performed with (6), so that data are centred on 0 and properly scaled. μ is the mean and σ is the standard deviation.

$$xstd = \frac{x - μ}{σ} \qquad ( 6 )$$

Label encoding is needed because the scikit-learn library [F. Pedregosa et al., “Scikit-learn: Machine learning in Python,” J. Mach. Learn. Res., vol. 12, pp. 2825-2830, 2011], which was used in Python for the present research work, only handles real numbers. Integers from 0 to 9 were used to encode the labels in the dataset. The data were then segmented in non-overlapping windows of 200 samples in length, equal to 2 seconds of recording.

After these steps splitting into training and test sets was required. It was chosen to use 80% of the data for the training set and the remainder 20% for the test set. The seed to the random generator was set equal to 42.

### 1) Machine Learning Methods

In machine learning methods the feature extraction must be performed before the model training. The selected features are both in time domain and frequency domain for one unit, while only the ones in time domain were used for the three units.

The time domain features were extracted from the time series of the signal and are the following: mean, standard deviation, variance, kurtosis, skewness, peak-to-peak distance, median, interquartile range. The frequency domain features were extracted from the Fast Fourier Transform (FFT) of the signal and are the following: mean, standard deviation, skewness, maxima and minima of the FFT, mean and maximum of the power spectral density.

Three machine learning methods were used: a K-Nearest Neighbor classifier (KNN), a Random Forest classifier (RF) and a Support Vector Machine (SVM).

In the case of the KNN classifier, the metric chosen for the computation of the distance was the Euclidean metric (L. E. Peterson, “K-nearest neighbor,” Scholarpedia, vol. 4, no. 2, p. 1883, 2009). The optimal number of neighbors K is around 5, since afterwards the accuracy score decreases.

In the case of the RF classifier (C. Vercellis, Business intelligence: data mining and optimization for decision making. Wiley Online Library, 2009), the splitting rule to create the nodes of the trees that compose the forest is the Gini Criterion. Afterwards, in each node the corresponding attribute is chosen by minimizing the impurity, as it is traditionally done with RF classifiers.

Since the data of this research project could not be separated linearly in the original space, to develop a SVM a kernel was used. In this case, it was decided to use the Radial Basis Function Kernel, which can be expressed mathematically as (7):

$$K ( K 1 , K 2 ) = exp ( - \frac{{‖ X 1 - X 2 ‖}^{2}}{2 {σ}^{2}} ) \qquad ( 7 )$$

Where σ is the variance and the hyperparameter |X1-X2| is the Euclidean distance between two points X1 and X2. In this case, distance is used as an equivalent of dissimilarity: when the distance between the points increases, they are less similar. By default, σ is taken equal to one, so the kernel is represented by a bell graph, that decreases exponentially as the distance increases and is 0 for distances greater than 4.

### 2) Deep Learning Methods

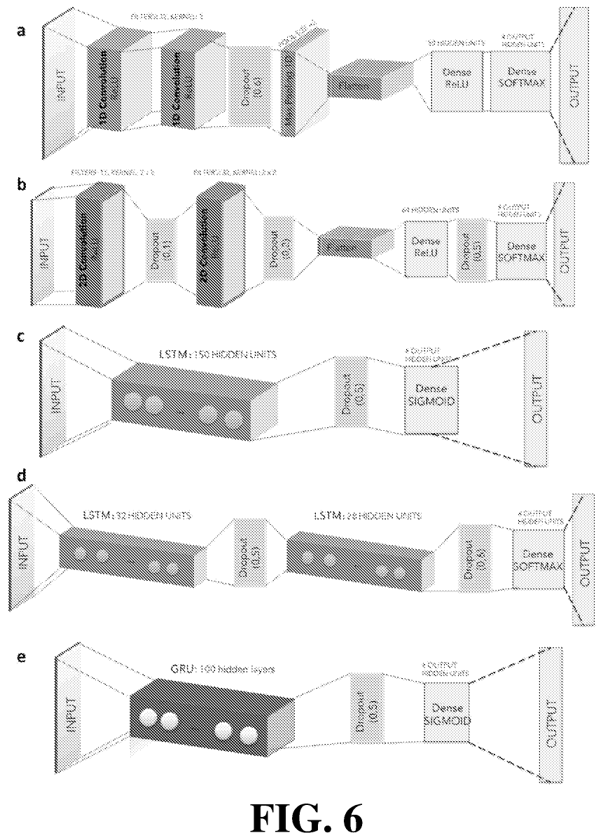

Five networks were created using deep learning methods; their characteristics are shown in detail in FIG. 6. The networks used were the following: a 1D Convolutional Neural Network (1DCNN), a 2D Convolutional Neural Network (2DCNN), a single-layer Long Short Term Memory (BASE LSTM), a multi-layer Long Short Term Memory (MULTI LSTM) and a Gated Recurrent Unit (GRU).

All networks use the Sequential Model in order to construct a plain stack of layers where each layer has exactly one input tensor and one output tensor. During the optimization part of the algorithm, the error at the current state must be iteratively estimated. For all networks, the chosen loss function was the Sparse Multiclass Cross-Entropy Loss as in (8), used to calculate the model's loss such that the weights can be updated to minimize the loss on subsequent evaluations. The Cross-Entropy loss is defined as:

$$J ( W ) = - {\sum }_{i = 1}^{N} {y}_{i} log ( {{y}^{⌢}}_{i} ) \qquad ( 8 )$$

Where w refers to the model parameters, yᵢ is the true label and ŷᵢ is the predicted label.

After that, to reduce the losses, an optimizer was used to adjust the neural network's attributes such as weights and learning rate. The optimization method for the CNNs and GRU used was the Adam optimizer (D. P. Kingma and J. Ba, “Adam: A method for stochastic optimization,” arXiv Prepr. arXiv1412.6980, 2014) based on adaptive estimates of lower-order moments. For the LSTM networks, the RMSprop optimizer was used. The batch size is a hyperparameter that defines the number of samples taken from the training dataset to train the network before updating the internal model parameters; the chosen value was 16.

### III. RESULTS

### A. Respiratory Rate

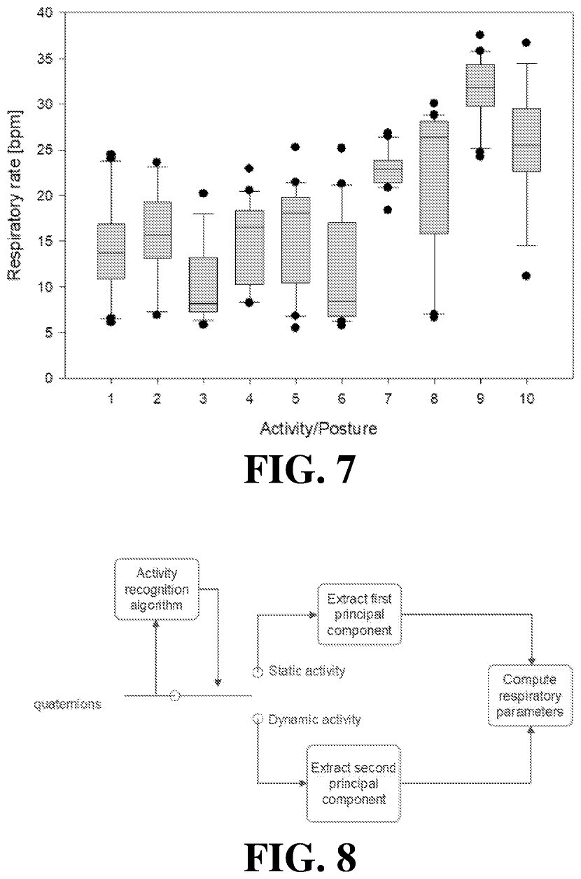

Respiratory rate was studied for the 20 involved subjects in the different postures and activities. Due to the unfavorable signal-to-noise ratio, parameters could not be extracted in the case of climbing stairs with the previously validated algorithm, therefore those values are not included in the analysis. The dataset presented puts together the two sitting positions, but separates “walking slow” and “walking fast” to show the sensitivity of the respiratory analysis algorithm to the different levels of effort. The boxplots of the median values obtained for each subject in the different conditions are shown in FIG. 7.

The distributions were statistically compared one with the other with the One-Way Repeated Measurements ANOVA. The Shapiro-Wilk normality test was failed (p<0.05); also the Equal Variance Test (Brown-Forsythe) was failed (p<0.05). The differences in the mean values among the groups were greater than would be expected by chance; there is a statistically significant difference (p≤0.001).

To isolate the group or groups that differ from the other, the Bonferroni t-test was used as multiple comparison procedure. The p-values obtained with these comparisons were analyzed. The activities, “walking slow”, “walking fast”, “running” and “cycling” have a statistically significant difference with respect to static postures (p<0.05 in all cases), but not always one with respect to the other. “Walking slow” and “walking fast” do not significantly differ from “cyclette” (p=1.000) and between one another (p=1.000). This result confirms that during physical activity the respiratory rate increases and this phenomenon is more evident when the activity is more demanding (during “running”). Also, there is a statistically significant difference between the “supine” and the “prone” positions (p=0.045) and between the “lying right” and the “prone” positions (p=0.049). This is likely due to the fact that the processing algorithm is designed to analyze the movement of the two units in the front with respect to the reference unit, while in prone position also the dorsal movement contributes to ventilation.

### B. Human Activity Recognition Algorithm

All methods with three units have better performances when compared to the case with only one unit. The most performing one is the 1D Convolutional Neural Network (1DCNN) with three units. It has to be considered also that the features extracted for the three units are only concerning time, which suggests that the inclusion of the frequency features could further improve the accuracy.

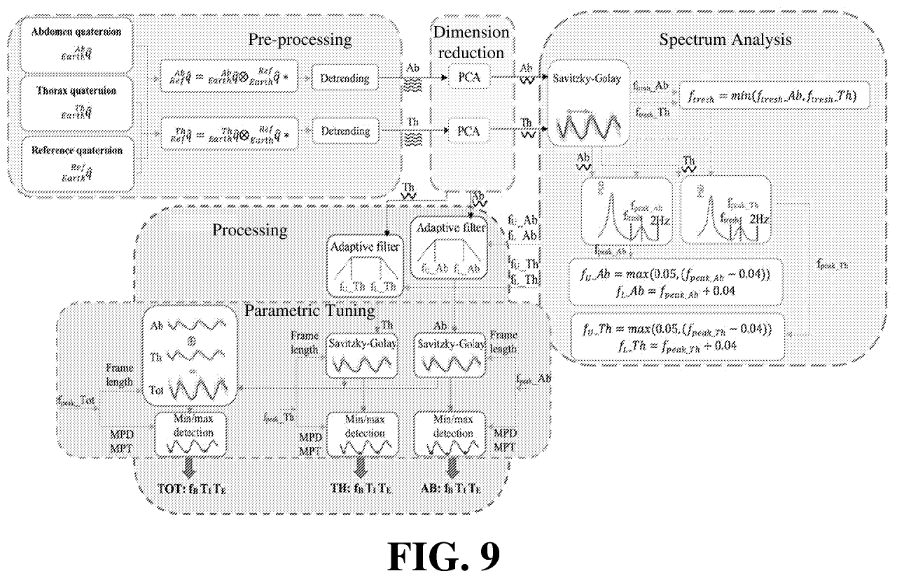

A block diagram of an algorithm for estimating respiratory parameters of a person is schematically illustrated in FIG. 9 and is discussed in detail in [A. Cesareo, Y. Previtali, E. Biffi, and A. Aliverti, “Assessment of breathing parameters using an inertial measurement unit (IMU)-based system,” Sensors (Switzerland), vol. 19, no. 1, pp. 1-24, 2019, doi: 10.3390/s19010088]. This prior algorithm was based on the analysis of the first principal component of the quaternions representing signals from the inertial sensors installed on thorax and on abdomen referred to the reference quaternion provided by the reference inertial sensor.

The required steps to calculate the principal components are the following:
standardize input data, because all values need to have the same range of variation. In the case of quaternions, they are already between −1 and 1 so no standardization is necessary;
compute the covariance matrix to understand how the variables of the input data set are varying from the mean with respect to each other;
compute eigenvectors and eigenvalues of the covariance matrix. Eigenvectors are the directions of the axes where there is the most variance (most information), so they are the principal components. Eigenvalues are the coefficients attached to eigenvectors, which give the amount of variance carried in each principal component. By ranking eigenvectors in order of their eigenvalues, highest to lowest, principal components in order of significance are obtained.

For static activities the first principal component of inertial sensors installed on thorax and abdomen (referred to the reference sensor) is taken because respiratory signal is thought to be lying in it. For dynamic activities such as walking or running, it is observed that the first principal components contain information about movement (such as cadence) and, in order to retrieve the respiratory signal, higher order principal components may be considered. It has been noticed that the first principal component contains information about movement and the second principal component contains information about the respiratory signal, but in general the respiratory component during dynamic activities may be found in higher order principal components than the first principal component.

According to this disclosure, it has been tried to apply the algorithm shown in FIG. 8 to estimate respiratory parameters using the first principal component, and it has been repeated using the second principal component in order to assess whether or not more reliable estimations may be obtained by using the second principal component analysis. The following Table 1 compares values estimated by a device (K5), used as a gold standard for estimating respiratory parameters, and values estimated using the algorithm of FIG. 8 using the first principal component analysis and the second principal component analysis:

TABLE 1

| | | | 1° Principal | | | 2° Principal | | |
|---|---|---|---|---|---|---|---|---|
| | | K5 | Component | | | Component | | |
| Condition | Breath | frequency ± SD | Abd | Thor | Tot | Abd | Thor | Tot |
| Static | Spontaneous | 16.0 ± 1.9 | 7.2 | 15.0 | 14.6 | 7.5 | 7.5 | 7.4 |
| Walking | Spontaneous | 33.9 ± 4.2 | 59.8 | 69.4 | 60.0 | 31.6 | 33.3 | 31.6 |
| | Rise | 34.8 ± 5.0 | 61.8 | 59.4 | 60.0 | 31.6 | 33.3 | 32.5 |
| | frequency | | | | | | | |
| | (voluntary) | | | | | | | |
| | Decrease | 25.3 ± 7.7 | 59.8 | 60.1 | 60.1 | 35.3 | 27.3 | 36.4 |
| | frequency | | | | | | | |
| | (voluntary) | | | | | | | |
| Run 7 | Spontaneous | 36.1 ± 4.2 | 82.5 | 82.5 | 82.5 | 37.9 | 37.7 | 37.7 |
| km/h | | | | | | | | |
| Run 8 | Spontaneous | 35.6 ± 4.7 | 80.4 | 80.4 | 80.4 | 37.6 | 38.0 | 37.3 |
| km/h | | | | | | | | |

It was observed that, in static postures, reliable estimations may be obtained by processing the first principal component. By contrast, for dynamic activities such as walking and running, more accurate estimations may be obtained by processing the second principal component, whilst the first principal component contains information about the cadence of the dynamical activity. Moreover, it has been found that it is possible to obtain accurate estimations using only two inertial sensors, namely a single inertial sensor (preferably installed either on the thorax or on the abdomen) and the reference inertial sensor. Preferably, after having determined the type (static/dynamic) of a human activity in progress, the step of estimating respiratory parameters may be carried out with an inertial sensor installed on the thorax and with the reference inertial sensor. According to an alternative, the step of estimating respiratory parameters is carried out with an inertial sensor installed on the abdomen and with the reference inertial sensor.

Therefore, as shown in FIG. 8, it is possible to get accurate estimations of respiratory parameters by determining first whether the person is carrying out either a static activity or a dynamic activity, then by executing the algorithm of FIG. 9 over either the first principal component or the second principal component depending on whether a static activity or a dynamic activity is in progress.

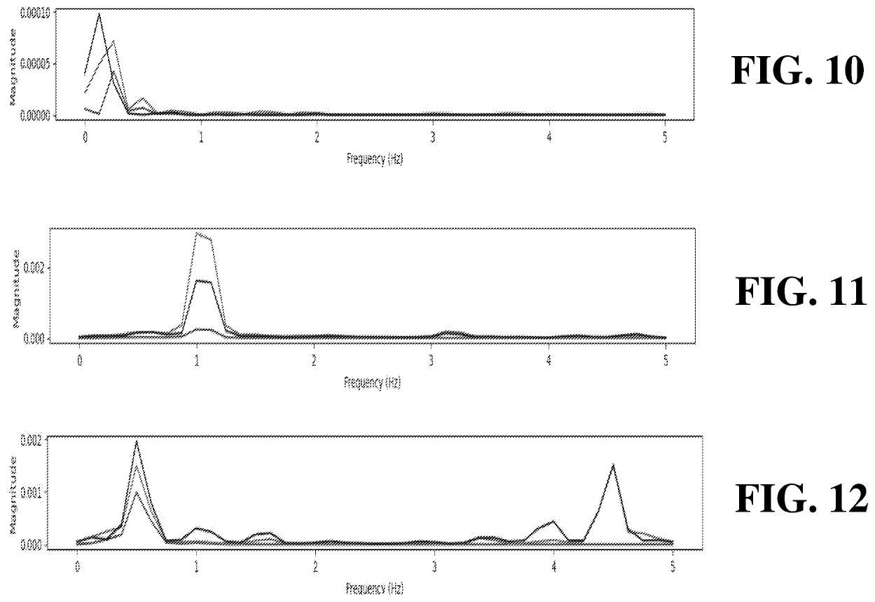

Merely as a comparative example, power spectral densities of the first principal components are represented in FIG. 10 in case of a static human activity and power spectral densities of the first and of the second principal components are compared in FIGS. 11 and 12 in case of a dynamic human activity. More in detail, FIG. 10 is a graph of the power spectral density of the first principal component in a static posture of signals of inertial sensors installed on the thorax and on the abdomen referred to the reference sensor, and the power spectral density of the sum of the two signals. The shown peaks are concentrated at very low frequencies, highlighting a low respiratory frequency that could be accurately measured.

FIG. 11 is a graph of the power spectral density of the first principal component during walking activity of signals of inertial sensors installed on the thorax and on the abdomen referred to the reference sensor, and the power spectral density of the sum of the two signals. The peaks are all around 1 Hz because the walk cadence was of about 120 steps/min and the inertial sensors detect only half of the actual cadence, i.e. the cadence determined every time the person is starting a step with the same foot. It is clear from these exemplary graphs that in this case the first principal component is mainly determined by the walk cadence and it is hard to derive accurate information therefrom about respiratory parameters of the person.

FIG. 12 is a graph of the power spectral density of the second principal component of signals of inertial sensors installed on the thorax and on the abdomen referred to the reference sensor, and the power spectral density of the sum of the two signals, during the same walking activity as for FIG. 11. The peaks of the power spectral densities of the second principal components of the signals generated by the inertial sensors (referred to the reference sensor) installed on the thorax and on the abdomen are concentrated at about a frequency of 0.5 Hz because the respiratory rate in this test example was of about 30 breaths per minute. The considered walking activity, as well as other dynamic activities, required an upright posture of the person, thus the thoracic signal generated by the inertial sensor installed on the thorax is expected to be more reliable than the abdominal signal generated by the inertial sensor installed on the abdomen, because the latter is more subject to non-respiratory related movements.

As schematically represented in FIG. 8, estimations of respiratory parameters of a person may be enhanced by integrating the posture and activity recognition algorithm disclosed above. Quaternions may be used to first predict the activity with an artificial intelligence algorithm. The prediction is used to choose the proper principal component to be used in the algorithm shown in FIG. 9. If a static human activity is recognized as in progress, then the most valuable information about the respiratory signal is expected to be contained in the first principal component. On the other side, if a dynamic human activity is recognized, then the most valuable information about the respiratory signal is not in the first principal component, but in the second principal component or, more in general, in a higher order principal component (second, third, fourth . . . principal component). In the shown embodiments, only the second principal components have been taken into consideration because they provided the best results, though tests have shown that it is also possible to use other higher order principal components, in substitution or in addition to the second principal components of signals generated by the inertial sensors.

1. A method for continuous monitoring a respiratory rate of a person, comprising the steps of: determining an activity of a person, comprising the steps of:
positioning at least a first inertial sensor either on the abdomen or on the thorax of a person;
positioning a reference inertial sensor on a part of the body not subject to respiratory movements, fixed with respect to the torso;
wherein each inertial sensor supplies a signal represented by a quaternion that describes the orientation of said inertial sensor with respect to the Earth's reference system;
providing a fourth quaternion by referencing the orientation of a first quaternion to a reference quaternion, wherein said reference quaternion represents the spatial orientation of said reference sensor and wherein said first quaternion represents the spatial orientation of said first inertial sensor;
wherein it further comprises the steps of:
1) determining preliminarily whether an activity in progress of a person is either a static activity or a dynamic activity, without determining a respiratory rate of the person, by means of a pre-trained activity recognition algorithm receiving as entries at least said reference quaternion;
2) if at step 1) it is determined that said activity in progress is a static activity, then determining the respiratory rate from a first filtered replica of a first principal component of said fourth quaternion, wherein said first filtered replica is obtained by filtering said first principal component with a first filter;
3) if at step 1) it is determined that said activity in progress is a dynamic activity, then determining the respiratory rate either from:
a second filtered replica of said first principal component of said fourth quaternion obtained by filtering said first principal component with a second filter to be used only in case a dynamic activity has been determined, wherein said second filter is different from said first filter, or
a third filtered replica of a second principal component or of a higher order principal component of said fourth quaternion, wherein said third filtered replica is obtained by filtering with said first filter said second principal component or higher order principal component of said fourth quaternion.

2. The method according to claim 1, comprising the step of:
every time it is determined at step 1) that said activity in progress is a dynamic activity and not a static activity, determining the respiratory rate from said third filtered replica obtained by filtering, with said first filter, said second principal component or higher order principal component of said fourth quaternion.

3. The method according to claim 1, wherein each inertial sensor comprises an accelerometer, a magnetometer, and a gyroscope, and a microprocessor that receives the signals from said accelerometer, magnetometer, and gyroscope, and where each of said microprocessors processes said signals and supplies said signal represented by said quaternion that describes the orientation of said inertial sensor with respect to the Earth's reference system, the method further comprising the steps of:
sending said first quaternion representing the spatial orientation of said at least a first sensor to a control centre;
sending said reference quaternion representing the spatial orientation of said reference sensor to said control centre;
referencing the orientation of said first quaternion to said reference quaternion, to provide said fourth quaternion.

4. The method according to claim 1, further comprising the step of determining whether an activity in progress of a person is either a static activity or a dynamic activity by means of a pre-trained learning machine receiving as entries also said first quaternion.

5. The method according to claim 1, further comprising the following steps:
positioning a second inertial sensor either on the thorax or on the abdomen;
sending a second quaternion representing the spatial orientation of said second sensor to a control centre;
referencing the orientation of said second quaternion to said reference quaternion, to provide a fifth quaternion;
determining a first principal component of said fifth quaternion;
if it is determined that said activity in progress is a static activity, then determining a respiratory rate also from a fourth filtered replica of the first principal component of said fifth quaternion, wherein said fourth filtered replica is obtained by filtering the first principal component of said fifth quaternion with a third filter;
if it is determined that said activity in progress is a dynamic activity, then determining a respiratory rate also from either:
a fifth filtered replica of the first principal component of said fifth quaternion obtained by filtering the first principal component of said fifth quaternion with a fourth filter to be used only in case a dynamic activity has been determined, wherein said fourth filter is different from said third filter, or
a sixth filtered replica of a second principal component or of a higher order principal component of said fifth quaternion, wherein said sixth filtered replica is obtained by filtering with said third filter said second principal component or higher order principal component of said fifth quaternion.

6. The method according to claim 5, further comprising the step of determining whether an activity in progress of a person is either a static activity or a dynamic activity by means of a pre-trained learning machine receiving as entries also said second quaternion.

7. The method according to claim 1, wherein said step of determining an activity in progress of said person, is carried out by pre-trained activity recognition algorithm that determines upon said entries the activity in progress by choosing the activity in progress in a set composed at least of the following static activities: sitting, lying supine, staying prone, lying on a side, standing up; and composed at least of the following dynamical activities: walking slow, walking fast, running, cycling.

8. The method according to claim 7, wherein said pre-trained learning machine is a deep learning network chosen in the set composed of: 1D convolutional neural network, 2D convolutional neural network, single-layer long short term memory, multi-layer long short term memory, gated recurrent unit.
